(19) United States
(12) Patent Application Publication
(10) Pub. No.: US 2010/0063559 A1
(43) Pub. Date: Mar. 11, 2010

(54) SINGLE-USE EXTERNAL DEFIBRILLATOR

(76) Inventors: Allister R. McIntyre, Newtownards (GB); John McCune Anderson, Holywood (GB); Johnny Houston Anderson, Holywood (GB)

Correspondence Address: PORTER WRIGHT MORRIS & ARTHUR, LLP;INTELLECTUAL PROPERTY GROUP, 41 SOUTH HIGH STREET, 28TH FLOOR, COLUMBUS, OH, 43215, US

(21) Appl. No.: 12/514,670

(22) PCT Filed: Nov. 15, 2007
(86) PCT No.: PCT/EP2007/009879

§ 371 Date: May 27, 2009

(30) Foreign Application Priority Data
Nov. 15, 2006 (IE) S2006/0825

(51) Int. Cl. A61N 1/39

(52) U.S. Cl. 607/5

(57) ABSTRACT

A single-use battery-powered external defibrillator comprises a sealed defibrillator housing 10 containing a battery-powered defibrillator circuit and a pair of defibrillator electrodes 14 permanently attached by leads 22 to the circuit within the housing.

This invention relates to a single-use battery-powered external defibrillator.

The concept of using electrical energy as a means of ‘kick starting’ the heart toward recovery from Cardiac Arrest has been known since the 19th Century. However, the idea of producing a portable defibrillator, and the technology to realise such a device, has only been promoted from the early 1970's. Pioneered in the Royal Victoria Hospital in Belfast, Northern Ireland, the portable defibrillator was born of the need to restart the heart as soon after arrest as possible. By placing a defibrillator in the ambulance, the patient no longer had to be transported to a hospital before resuscitation could begin. It was recognised that this was often too late for either recovery or a good prognosis. Furthermore, the diagnosis of whether or not electrotherapy should be applied to a patient had to be conducted by a trained and fully qualified cardiologist.

Technology has advanced such that miniaturisation is now possible and it is now possible for software to analyse the ECG and determine whether or not the patient should be shocked. This means that the skills required to diagnose patient arrhythmia can be built into the defibrillator such that even the minimally trained can use the device safely and effectively. Clinical research, however, has led the way to, and still pursues, greater efficacy. The amount of electrical therapy applied to a patient is a compromise between applying sufficient energy to restart the heart and yet minimising that same energy to ensure that heart tissue is not damaged. In the early years of development, monophasic energy pulses of up to 400 joules were used (energy applied in a single direction) but research has shown that less energy of around 150 joules need only be applied if the pulse is biphasic; that is, can be split into two phases—current passed in one direction and then the other.

The current state of the art has produced external defibrillators which can be used by minimally trained personnel, incorporating either selectable or escalating energies from 150 joules to 250 joules and with a accuracy of diagnosis better than 99.6%.

In the pursuit of even greater ease of use by non-skilled persons, it would be desirable to provide a single-use external defibrillator, that is to say, a defibrillator intended for use, e.g. in the home or office, by minimally trained personnel on only one occasion, i.e. on a single patient. After use, or at the end of its shelf life, such a defibrillator could be disposed of or returned to the manufacturer for re-conditioning. For such a defibrillator the over-riding requirement will be for safety, effectiveness, reliability and low-cost in a long storage environment.

The design and implementation of a single-use external defibrillator imposes certain problems hitherto unaddressed by prior art re-usable defibrillators.

Existing external defibrillators use removable electrodes. The problems arising with removable electrodes are:
The contact between the electrodes and the defibrillator proper has to accommodate the passage of high current, high voltage electrical discharge to the patient and also low voltage, ultra-low current monitoring signals. Such parameters require widely differing physical solutions and a compromise can only ever be implemented.
The operation of attaching the electrodes to the defibrillator represents one further action required of the operator possibly delaying the application of therapy to the patient. Likewise, any removable part requires training of the user so that the part can be inserted correctly with true alignment and engagement.
The electrode pack can be mislaid or out of stock without the operator realising until the need for the device is apparent.

Prior art external defibrillators use a removable and replaceable battery. The problems associated with such batteries are:
It is necessary to monitor the battery to ensure that there is sufficient charge remaining in the battery for the next use. If it is possible for the battery to be transferred into another device, then the battery usage history must also be transferred.
The contact of the battery-defibrillator interface has to accommodate both high and low currents which can dictate different contact materials. The contact material must therefore be a compromise and cannot be ideal.

Another problem with defibrillator electrodes is that their characteristics change with time. One on the most critical characteristics is the AC impedance—the resistance of the gel itself. A significant electrode AC impedance means that energy can be lost in the electrode gel instead of being applied to the patient.

An object of the present invention is to provide a single-use defibrillator in which the above disadvantages are avoided, mitigated or eliminated.

According to the present invention there is provided a single-use battery-powered external defibrillator comprising a sealed defibrillator housing containing a battery-powered defibrillator circuit and a pair of defibrillator electrodes permanently attached by leads to the circuit within the housing.

By “sealed” we mean that the housing, once closed by the manufacturer, cannot be re-opened by the casual user without visible damage to the housing (although it might be possible for the manufacturer to open it using specialised tools). It does not imply that the housing is hermetically sealed. By “permanently attached” we mean that the electrodes cannot be detached from the housing except by severing the leads or the electrodes.

The advantage of having a sealed housing is that it does not allow the battery to be replaced by the operator. The battery capacity is known and the history of usage need not be transferred to another device but can be held within internal memory. Also, the battery can be directly connected, e.g. by solder, to the defibrillator circuitry, affording the optimal solution to minimum contact resistance to all currents.

The advantage of having permanently attached electrodes, e.g. by soldering the leads to the internal defibrillator circuitry, are that the current path can be made suitable for both high current and low current transfer at any voltage. Further, there is no delay or possibility of error in making a connection to the device and no need for special training in the operation. Finally, since the electrodes are permanently attached, there is no need to ensure that the electrode pack is in place and within its use-by date.

A further advantage of permanently attached electrodes is that it affords a solution to electrode aging. Due to the electrodes being permanently attached their age is known and, since the change of AC impedance with time is relatively fixed and known, it is possible for the defibrillator to compensate for the known impedance change to ensure that the patient receives the correct energy over the lifetime of the electrodes. It should be noted that the other primary characteristics change little with time and the AC impedance parameter almost exclusively defines the lifetime of the electrodes.

Accordingly, in a preferred embodiment, in order to compensate for changes in electrode impedance over the lifetime of the electrodes the peak voltage delivered to the patient, and/or the duration of the two phases of a biphasic waveform delivered to the patient, is increased as a function of the age of the electrodes.

Another problem with defibrillators is the high current needed to charge the capacitors prior to discharge. The prior art teaches a need for ‘dump’ resistors such that any charge remaining on the capacitors either before or after therapy delivery can be ‘dumped’ into resistors, dissipating the energy and discharging the capacitors.

In the preferred embodiment of single-use defibrillator any capacitor charge is not dumped but any excess charge is allowed to dissipate through the natural leakage of the capacitors and the voltage monitoring circuit. As a result, successive requirements for therapy need only ‘top-up’ the charge rather than charge from zero. The advantages are a shorter charge time and a decrease in battery consumption which has a direct impact on the size of the batteries and hence the overall size of the defibrillator housing.

In the preferred embodiment, too, the battery is intermittently tested by initiating a short capacitor charging cycle and measuring the magnitude of the resulting dip in battery voltage.

In a defibrillator a significant size and weight reduction is made possible by a reduction in the energy delivered to a patient. Such an energy reduction permits smaller, lighter capacitors and batteries. However, a reduction in energy to the patient is only made possible if it can be shown to be as effective in cardiac resuscitation as the higher energies. Our extensive research has determined, surprisingly, that if the output energy pulses have limited tilt (voltage drop from the beginning to the end of the pulse), a lower energy can be used. This feature is used in the present embodiment of the invention, but is applicable to defibrillators generally.

Accordingly, there is also provided, as a further and separate invention, an external defibrillator comprising means for applying to a patient a biphasic energy pulse in which each discharge pulse has a tilt of less than 25% and a duration of 3-10 ms, and the peak voltage is less than 1300 volts.

An embodiment of the invention will now be described, by way of example, with reference to the accompanying drawings, in which:

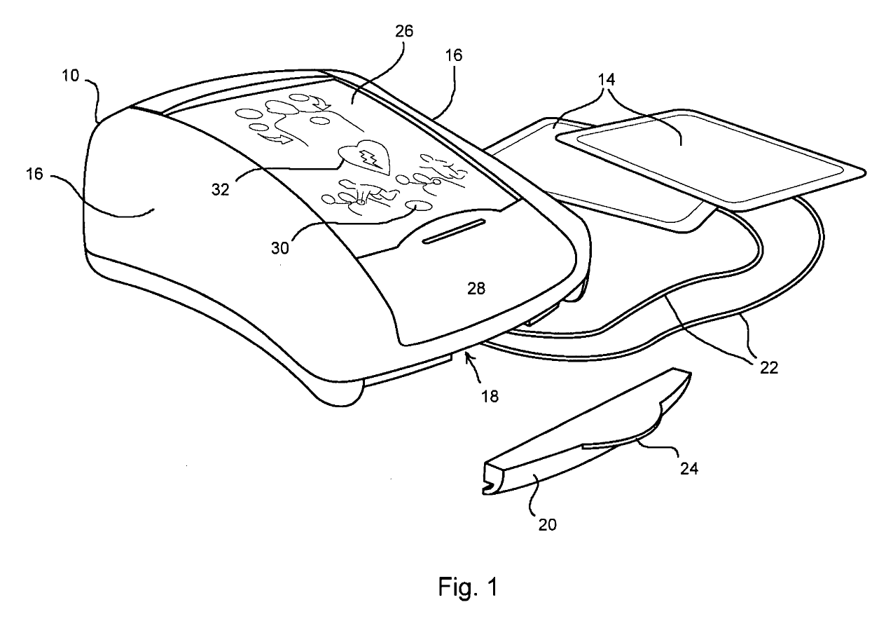

FIG. 1 is a perspective view of an external defibrillator according to the embodiment with its electrodes deployed for use.

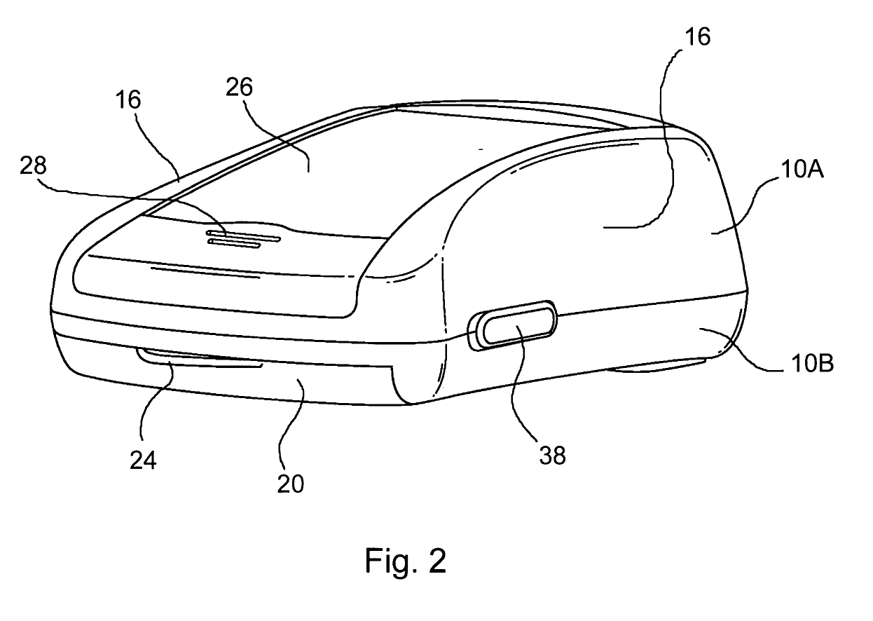

FIG. 2 is a perspective view of the defibrillator of FIG. 1 with its electrodes stowed.

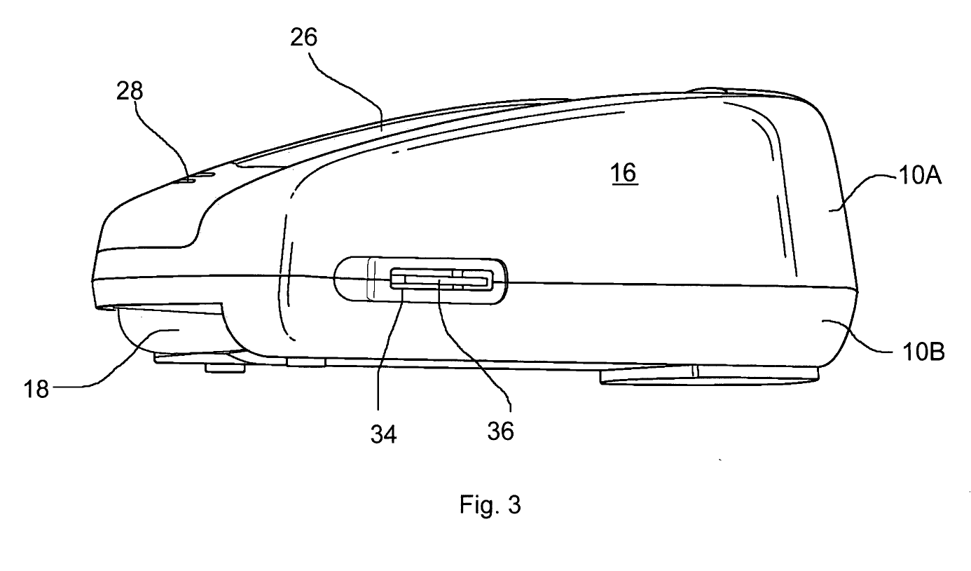

FIG. 3 is a perspective side view of the defibrillator of FIG. 1 with its electrodes omitted and its memory card bay exposed.

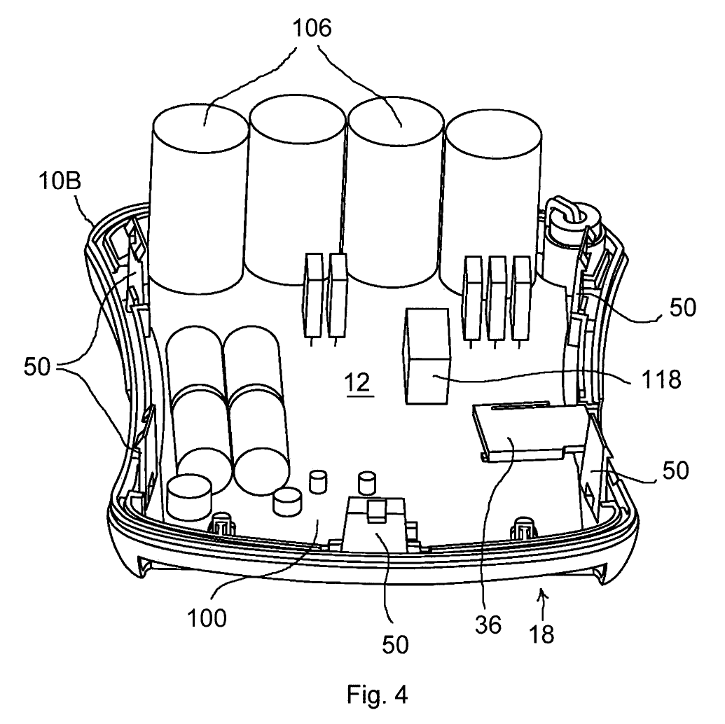

FIG. 4 is a perspective front view of the bottom half of the defibrillator housing showing the internal components.

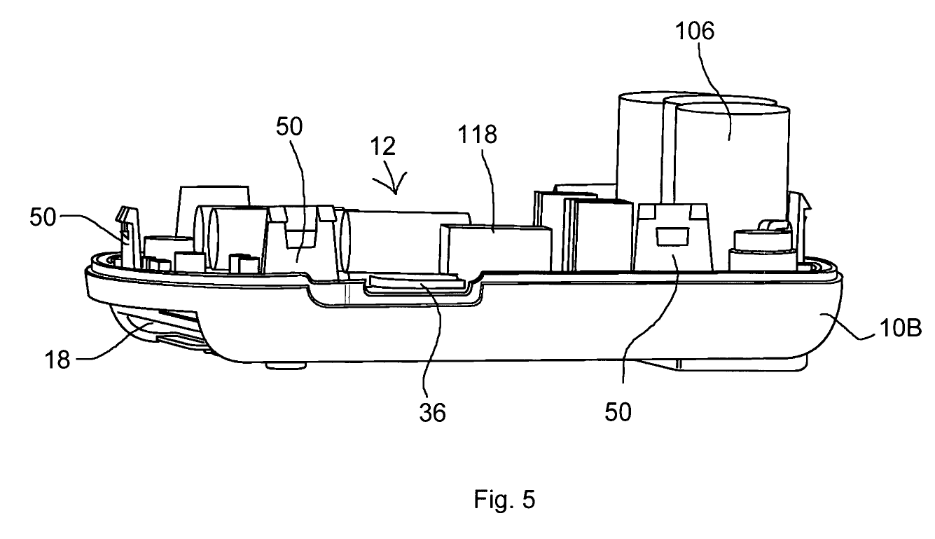

FIG. 5 is a perspective side view of the bottom half of the defibrillator housing showing the internal components.

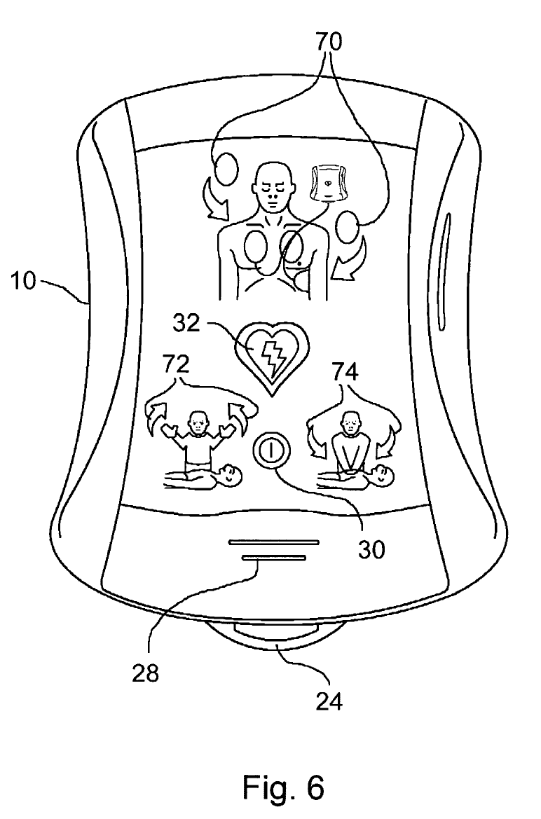

FIG. 6 is a top plan view of the defibrillator showing details of the keypad.

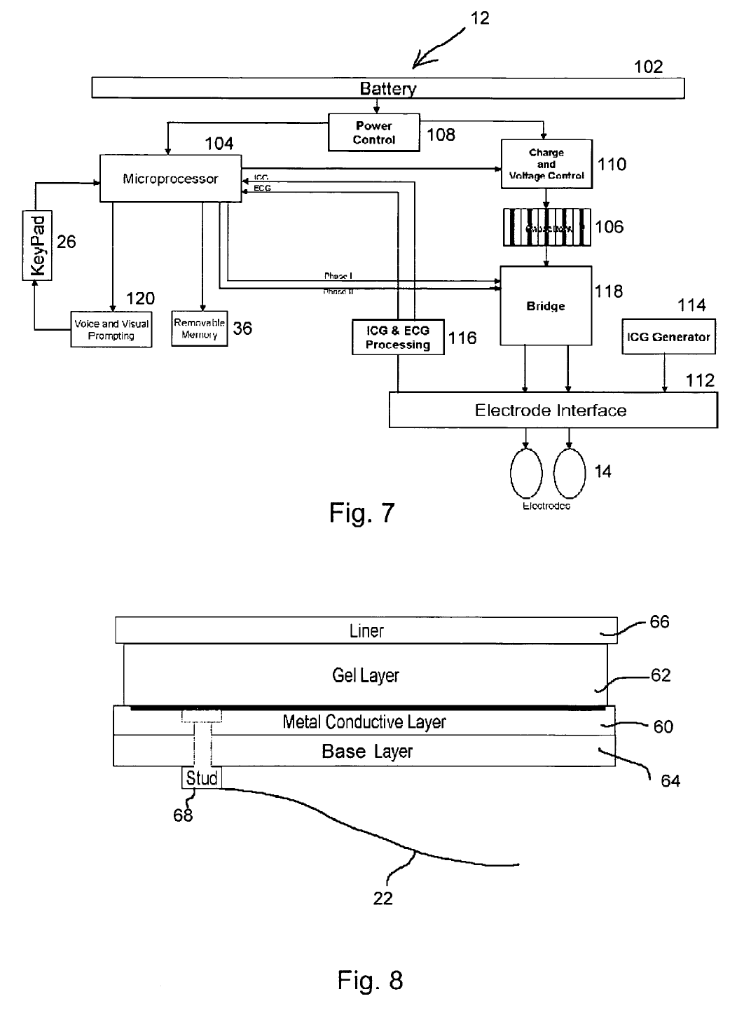

FIG. 7 is a block diagram of the defibrillator circuitry.

FIG. 8 is a diagrammatic cross-section of one of the defibrillator electrodes.

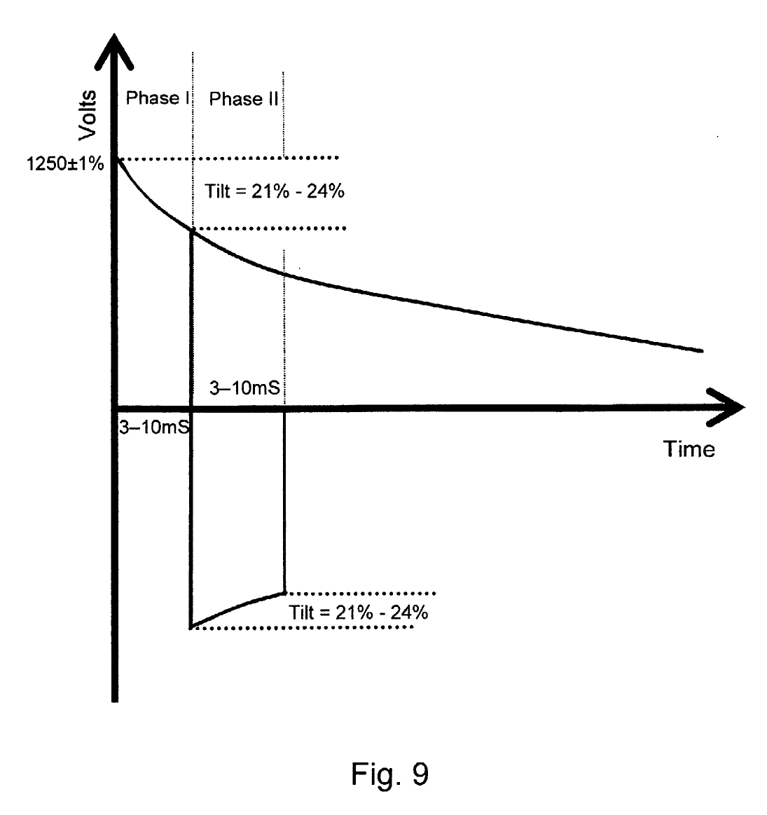

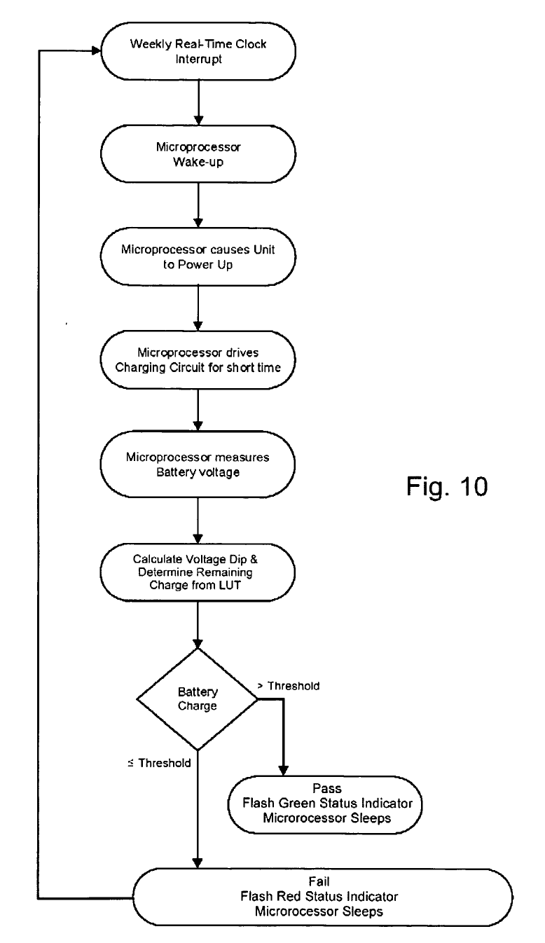

FIG. 9 is a waveform diagram showing the biphasic energy delivered to the patent.

FIG. 10 is a flow diagram of the battery self-test function of the defibrillator circuit.

Referring to the drawings, a single-use portable automated external defibrillator comprises an outer housing 10 containing the main defibrillator circuitry 12 (FIGS. 4, 5 and 7). The housing 10 is designed for grasping in one hand while withdrawing the defibrillator electrodes 14, to be described, with the other hand and applying the electrodes to the patient. To achieve this, the housing 10 has non-slip surfaces 16 on opposing planes at a distance less than the span of a human hand.

Then defibrillator has two electrodes 14 which are connected by respective leads 22 to the internal defibrillator circuitry 12. The defibrillator electrodes 14 are normally stowed in a bay 18 at the front of the housing 10, the bay being closed by a removable front cover 20. During manufacture, the electrodes 14 are placed in the bay with the coiled leads 22. The electrodes are physically connected to the cover 20 such that, when the user pulls the tab 24 on the cover, the electrodes 14 are automatically released and the leads uncoiled as the cover is removed.

The top surface of the housing 10 has a keypad 26 and a speaker 28. The keypad 26 has, inter alia, an “ON/OFF” button 30 and a manual “SHOCK” button 32, FIG. 6. After turning on the defibrillator, voice prompts and flashing symbols on the keypad guide the user through the entire operational sequence through to the pressing of the button 32. The voice prompts may be complemented by visual indicators (not shown). If cardiopulmonary resuscitation (CPR) is required, the rate at which compressions should be applied is indicated by an audible click supported by flashing indicators. This is particularly important in assisting the lay user.

On one side the housing has a bay 34 for a removable memory card 36, FIG. 3. The bay 34 is normally closed by a removable cover 38, FIG. 2, allowing the memory card to be withdrawn. The memory card contains a record of the ECG, ICG and events which occurred during the deployment of the defibrillator. It can be returned from the manufacturer or distributor for a permanent record of the incident. The replaceable memory card can also be used for updating the defibrillator software (control program and algorithm) or for uploading software configuration data.

The internal circuitry 12 of the defibrillator is mounted on a circuit board 100, FIG. 4, and is powered by a battery 102 (FIG. 7) located under the circuit board 100. The operation of the circuitry is controlled by a microprocessor 104. In use, the electrodes 14 are deployed and attached to the patient. When the ‘ON/OFF’ button 30 is pressed, the device powers up and the patient impedance is measured to ensure that the electrodes 14 are attached correctly and to define, using an energy look-up table (LUT) in the microprocessor software, the voltage to which capacitors 106 should be charged as a function of the measured impedance of the patient and the duration of the biphasic pulses. The capacitors 106 are charged to this voltage by power control and charge and voltage control circuits 108, 110 and the ECG is continually monitored through an electrode interface circuit 110 after processing by signal amplification and conditioning circuits 116. At the same time a high frequency is generated by an ICG generator 114 and fed as a constant current to the patient, the ensuing voltage being processed by the signal amplification and conditioning circuits 116 and the generated ICG signal fed to the microprocessor 104. The ECG and ICG signals are examined by a diagnostic algorithm embedded in the microprocessor 104. If a shockable rhythm is diagnosed, the user is prompted to push the SHOCK button 32 whereupon the charge on the capacitors 106 is released, under control of the microprocessor 104, in two phases (Phases 1 & II—biphasic) by a high-voltage bridge 118 and applied to the patient through the electrodes 14. The duration of each phase is controlled by the microprocessor 104 in accordance with the LUT. As mentioned, the user is guided through the by voice and visual prompts 120.

FIG. 6 is a detailed view of the keypad 26. The SHOCK button 32 is heart-shaped and orange in colour. There is also a light behind the button. This button is enabled only when the device is charged and a shock is advised, at which time the button illuminates and flashes. When pressed by the user, the shock is delivered to the patient. The ON/OFF button 30 is green and is used to switch on the device. It will also allow the user to switch off the device at any time but issues a warning and has to be pressed again before the device will switch off. If the device is kept on for an inordinately long time, it will automatically switch off. The pad symbols 70 around the top figure of man will flash to indicate that the user needs to attach the electrodes to the patient. They will continue to flash until the device senses the attachment by measuring the impedance across the electrodes. When attached, the pad symbols 70 will cease to flash and the arrows 72 around the kneeling figure with patient will flash. These arrows 72 flash to indicate that the user must not touch the patient because it is analysing the patient's ECG or because a shock is about to be delivered. The arrows 74 around the user pressing down on patient's chest flash when CPR is advised—chest compressions and breathing therapy. Each of the above activities is accompanied by appropriate voice prompts.

In a defibrillator a significant size and weight reduction is made possible by a reduction in the energy delivered to a patient. Such an energy reduction permits smaller, lighter capacitors and batteries. However, a reduction in energy to the patient is only made possible if it can be shown to be as effective in cardiac resuscitation as the higher energies. We have found, surprisingly, that if the output energy pulses have limited tilt (voltage drop from the beginning to the end of the pulse), a lower energy (typically 120 joules) is as effective as the higher energy (typically 150 joules) conventionally used. Since the voltage reduces as a square law with energy, even this 20% reduction significantly reduces the capacitor and battery size. This feature is used in the present embodiment of the invention.

FIG. 9 is a waveform diagram of the biphasic pulses delivered by the present embodiment. According to the impedance of the patient, each discharge pulse has a tilt of less than 25%, preferably 21-24%, each discharge pulse has a duration of 3-10 ms, and the peak voltage on the capacitors is less than 1300 volts, preferably 1230-1280 v. This compares to a tilt of up to 50%, a pulse duration of 8-12 ms, and a peak voltage of 1650 v or greater for the prior art. The particular combination used in any particular case will be defined by the energy look up table for the patient impedance concerned. The low tilt is made possible by using capacitors with a total capacitance of 250 μF, compared to 120 μF for the prior art. We have found that using a reduced peak voltage and lower tilt allows the use of energies substantially lower than the prior art, typically around 120 joules.

The housing 10 is assembled from top and bottom “halves” 10A and 10B respectively, each moulded from a plastics material (only the bottom half 10B is shown in FIGS. 4 and 5). The bottom housing half 10B has a set of integral resilient clips 50 disposed at intervals around its internal periphery. The top housing half 10A has complementary ribs (not shown) likewise disposed around its internal periphery. When the top half 10A is fitted to the bottom half 10B the clips 50 snap-engage corresponding ribs to hold the two halves tight together with the clips then inaccessible. Thus the two housing halves are sealed against casual opening by the user, the only way to open the housing being to break the clips. Therefore, since the battery 102 is not user-replaceable, its terminals can be permanently soldered to the defibrillator circuitry with the advantages heretofore mentioned. Instead of using clips 50, the housing 10 can be sealed, for example, by ultrasonic welding or using a permanent adhesive.

In addition, the electrodes 14 are permanently attached by the leads 22 to the defibrillator circuitry 12, e.g. by soldering. This allows any change to the parameters of the electrodes over time to be compensated for by an adjustment in the peak voltage to which the capacitors 106 are charged and/or the duration of the two discharge phases.

The electrodes 14 comprise a metal conductive layer 60, a conductive gel layer 62, a non-conductive backing layer 64 and a liner 66, as shown in FIG. 8. In use the liner 66 is peeled off and the gel layer 62 is placed on the bare skin of the patient. The electrotherapy is applied through the lead 22 and a stud 68 to the metal layer 60 and dispersed across the gel layer 62 to the patient.

Due to the migration of ions across the gel-metal 60/62 interface and a slight loss of moisture from the gel, the electrical parameters of the gel layer can change with time. One such parameter is the AC impedance which affects the ability of the gel to pass current. If the AC impedance is high, then there will be a significance loss in energy in the electrode which will not be delivered to the patient. Over the lifetime of the electrodes, the change in electrode AC impedance can be up to 3 ohms and this rate of change can be measured and is substantially the same for all gel electrodes built to the same specification. This energy loss can be compensated by increasing the nominal energy to be delivered to the patient slightly above that actually required to be delivered, such that the patient receives the required energy after losses. However, this is only possible when the electrode age is known. This mandates that it must be effectively impossible for the user to remove the electrodes other than by destroying them or the connecting lead, since otherwise the age of the electrodes cannot be guaranteed.

The permanent attachment of the electrodes therefore permits the change in the electrode characteristic to be compensated for automatically by the defibrillator. The electrode changes, primarily in their AC impedance, are built into the energy look-up table. When a shock is advised, the defibrillator charges to a voltage which, taken with the measured patient impedance, deliveries the correct energy as determined by the look up table. As the AC impedance of the electrodes changes with time, the look-up table is modified so that the change can be compensated for. More particularly, the peak voltage delivered to the patient (i.e. the voltage to which the capacitor is initially charged) and/or the duration of the two phases of the biphasic waveform is increased as a function of the age of the electrodes. The change in impedance of the electrodes can, depending on patient impedance, represent a loss of energy from 2% to 12% of the energy delivered. Compensating for this loss can stabilize the energy delivered to the patient to within 2% across the range of patient impedances. Further, because of this compensation, it is possible to extend the life of the electrodes by ensuring that the patient receives the correct energy even if the impedance change is significant.

As a further departure from the prior art, after a shock has been given any charge remaining on the capacitors 106 is held pending a possible recharge if the therapy is unsuccessful and needs to be repeated. If no shock is advised, the charge on the capacitors 106 is not dumped but held pending a change in the patient's condition during monitoring. The time to recharge the capacitors for any subsequent requirement for the delivery of therapy is therefore greatly reduced.

The preferred embodiment is designed for a shelf life of greater than 5 years in ordinary circumstances. However, such is the effect and unpredictability of the storage temperature and other conditions under which the defibrillator could be stored or used, it is important to establish that the charge on the battery is adequate prior to an emergency occurring. Failure of the device to deliver the required shocks can result in failure to resuscitate a patient and, therefore death.

The batteries used in the present embodiment are lithium manganese dioxide which have particular characteristics:
Their charge decreases with temperature but neither linearly nor with much degree of accuracy and repeatability.
They have an internal resistance such that when a high current is drawn, the output voltage decreases abruptly but recovers quickly.
They have a small but reasonably predictable self-discharge which can increase with temperature.

These features can be used to determine the charge remaining in the battery. By testing a large number of batteries, a table can be draw up which correlates the voltage dip with the charge remaining on the battery. If this table is stored in a microprocessor and a battery subjected to a test with the result compared to the table, the charge remaining on the battery can be quite accurately determined.

Accordingly, in the preferred embodiment, a battery self-test is initiated by a pulse emitted by a real-time clock which is powered continuously by an on-board coin cell, designed to last many more times the life of the defibrillator (e.g. 17 years). Once a week, the real-time clock sends this pulse to the microprocessor which responds by performing an initialisation routine. This routine sends a signal to the power switch circuitry which causes the full battery power to be switched to all the device electronics. Among a number of other tasks the microprocessor then performs a check on the charge remaining in the battery by initiating a capacitor charging cycle. This charging cycle is very short, typically 100 mS, compared to a full charging cycle of about 12 secs, so that the battery is not significantly drained by the test.

During this very short charging cycle, the battery voltage will dip sharply and the magnitude of this dip is representative of remaining battery charge. The microprocessor measures this dip (using A-D conversion) and compares it to a table of values stored in flash memory.

From this, the remaining charge is determined and therefore the number of shocks available. If less than 10, a status indicator is flashed red to warn that the battery is low.

Since this is a single-use defibrillator, this, in effect, means that the unit must be replaced.

The corresponding flow diagram is shown in FIG. 10.

The invention is not limited to the embodiments described herein which may be modified or varied without departing from the scope of the invention.

1. A single-use battery-powered external defibrillator comprising a sealed defibrillator housing containing a battery-powered defibrillator circuit and a pair of defibrillator electrodes permanently attached by leads to the circuit within the housing.

2. A defibrillator as claimed in claim 1, wherein the peak voltage delivered to the patient, and/or the duration of the two phases of a biphasic waveform delivered to the patient, is increased as a function of the age of the electrodes.

3. A defibrillator as claimed in claim 1, further comprising means for applying to a patient a biphasic energy pulse in which each discharge pulse has a tilt of less than 25% and a duration of 3-10 ms, and the peak voltage is less than 1300 volts.

4. A defibrillator as claimed in claim 1, further comprising means for intermittently testing the battery by initiating a short capacitor charging cycle and measuring the magnitude of the resulting dip in battery voltage.

5. A defibrillator as claimed in claim 2, further comprising means for intermittently testing the battery by initiating a short capacitor charging cycle and measuring the magnitude of the resulting dip in battery voltage.

6. A defibrillator as claimed in claim 3, further comprising means for intermittently testing the battery by initiating a short capacitor charging cycle and measuring the magnitude of the resulting dip in battery voltage

7. A single-use battery-powered external defibrillator comprising a sealed defibrillator housing containing a battery-powered defibrillator circuit and a pair of defibrillator electrodes permanently attached by leads to the circuit within the housing; wherein the peak voltage delivered to the patient, and/or the duration of the two phases of a biphasic waveform delivered to the patient, is increased as a function of the age of the electrodes;
means for applying to a patient a biphasic energy pulse in which each discharge pulse has a tilt of less than 25% and a duration of 3-10 ms, and the peak voltage is less than 1300 volts; and means for intermittently testing the battery by initiating a short capacitor charging cycle and measuring the magnitude of the resulting dip in battery voltage.

8. A defibrillator as claimed in claim 2, further comprising means for applying to a patient a biphasic energy pulse in which each discharge pulse has a tilt of less than 25% and a duration of 3-10 ms, and the peak voltage is less than 1300 volts.
